(19) United States
(12) Patent Application Publication
(10) Pub. No.: US 2011/0124617 A1
(43) Pub. Date: May 26, 2011

(54) Combination Therapy for BreastCancer Comprising an Antiestrogenic Agent

(76) Inventors: C. Richard Lyttle, Bala Cynwyd, PA (US); Gary Hattersley, Stow, MA (US); Louis O'Dea, Hingham, MA (US)

(21) Appl. No.: 12/991,791

(22) PCT Filed: May 7, 2009
(86) PCT No.: PCT/US09/02885

§ 371 Date: Jan. 10, 2011

(30) Foreign Application Priority Data
May 9, 2008 (US) 61/127025

(51) Int. Cl. A61K 31/5685; A61K 31/138; A61K 31/4196; A61P 5/24; A61P 25/22

(52) U.S. Cl. 514/171; 514/647; 514/383

(57) ABSTRACT

This invention relates to combination therapies for the treatment of breast cancer comprising administering to a subject in need thereof a compound of Formula I or a pharmaceutically acceptable salt thereof and an anti-estrogenic agent (e.g., an aromatase inhibitor, a SERM that is not the SERM of Formula I, a GNRH agonist, a GNRH antagonist, or an estrogen receptor downregulator) and to compositions (e.g., pharmaceutical compositions) comprising a compound of Formula I or a pharmaceutically acceptable salt thereof and an anti-estrogenic agent. This invention also relates to a method of treating the side effects (e.g., vasomotor disturbances, osteoporosis and musculoskeletal complaints) associated with anti-estrogen therapy in a subject treated with one or more anti-estrogenic agents (e.g., an aromatase inhibitor, a SERM that is not the SERM of Formula I, a GNRH agonist, a GNRH antagonist or an estrogen receptor downregulator). The method comprises administering to the subject an effective amount of a compound of formula I or a pharmaceutically acceptable salt thereof.

### RELATED APPLICATION

This application claims the benefit of U.S. Provisional Application No. 61/127,025, filed on May 9, 2008. The entire teachings of the above application is incorporated herein by reference.

### BACKGROUND OF THE INVENTION

For decades, one of the mainstay treatments for breast cancer in humans has been the administration of tamoxifen and to a lesser extent, toremifene. Tamoxifen and toremifene are commonly referred to as Selective Estrogen Receptor Modulators (SERMs). The efficacy of the SERMs is putatively based on the ability to compete with endogenous estrogens (e.g., 17β-estradiol), thereby blocking the proliferative effects of these endogenous estrogens on mammary tissue. Both tamoxifen and toremifene are associated with side effects including hot flushes and stimulation of the endometrium in non-hysterectomized women, leading to an increase in uterine bleeding and uterine cancer. Interestingly, tamoxifen has been shown to confer a positive, estrogen-like benefit on the bone despite having an anti-estrogenic like effect on the breast.

More recently, aromatase inhibitors have become popular in the treatment of ER(Estrogen Receptor)-dependent breast cancers. Aromatase inhibitors work by blocking the conversion of precursor compounds (e.g., androstenedione) into estrogens, such as estrone. Popular aromatase inhibitors include both steroidal agents, such as exemestane, and non-steroidal agents, such as letrozole and anastrozole. Despite the growing acceptance of the aromatase inhibitors, they suffer from notable side effects including bone loss, increased bone fractures, vasomotor disturbances (e.g., hot flashes) and joint aches and pains. These effects are what one might expect given the induction of estrogen withdrawal that the agents are precipitating.

The combination of tamoxifen and anastrozole was included in the very large ATAC trial (Arimidex Tamoxifen Alone and in Combination trial). The combination arm of the trial (i.e., tamoxifen and anastrozole) was terminated early due to the failure to note any additional benefit relative to the tamoxifen monotherapy arm. By the conclusion of the study, anastrozole by itself appeared to be more effective at preventing breast cancer recurrence than tamoxifen. Despite the efficacy of aromatase inhibitors (e.g. anastrozole) and SERMs, such as tamoxifen and toremifene, both have serious issues for which combination therapy does not provide useful answers.

A common method for treating hormone-dependent conditions, such as hormone-dependent breast cancer or hormone-dependent prostate cancer, is to treat a patient with an agent that shuts down the endogenous production of sex hormones (e.g. estradiol and testosterone). These agents include gonadotropin releasing hormone agonists (GNRH agonists), such as buserelin, goserelin, histrelin, leuprorelin, nafarelin and triptorelin as well as gonadotropin releasing hormone antagonists (GNRH antagonists), such as abarelix, cetrorelix and ganirelix. While the efficacy of these agents can be very impressive due to their ability to cease hormonal production, they carry very strong and potentially harmful side effects. In this sense, their side effect profile might be considered to be similar to aromatase inhibitors (particularly in females), but with potentially greater magnitude due to the severity of the hormonal shut down. Again, for the same reasons spoken about with respect to aromatase inhibitors and SERMs, the ability to treat the side effects of GNRH agonists and GNRH antagonists is quite limited due to the fact that the most desirable treatment therapy, which comprises replacing the lost hormones, is contraindicated.

Clearly, new therapies are needed that can improve upon the side effect profile of one or more of these agents and/or increase their efficacy in treating breast cancer. The present application provides such therapies.

### SUMMARY OF THE INVENTION

This invention relates to combination therapies for the treatment of breast cancer comprising administering to a subject in need thereof a compound of

### Formula I

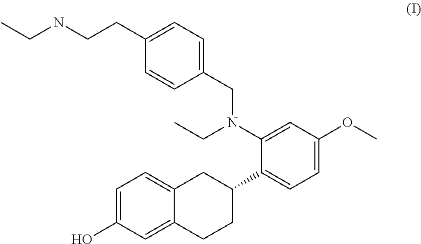

or a pharmaceutically acceptable salt thereof and an anti-estrogenic agent (e.g., an aromatase inhibitor, a SERM that is not the SERM of Formula I, a GNRH agonist, a GNRH antagonist or an estrogen receptor downregulator) and to compositions (e.g., pharmaceutical compositions) comprising a compound of Formula I or a pharmaceutically acceptable salt thereof and an anti-estrogenic agent. This invention also relates to a method of treating the side effects (e.g., vasomotor disturbances, osteoporosis and musculoskeletal complaints) associated with anti-estrogen therapy in a subject treated with one or more anti-estrogenic agents (e.g., an aromatase inhibitor, a SERM that is not the SERM of Formula I, a GNRH agonist, a GNRH antagonist or an estrogen receptor downregulator). The method comprises administering to the subject an effective amount of a compound of formula I or a pharmaceutically acceptable salt thereof.

More specifically, the invention described herein relates to compositions comprising a compound of formula I or pharmaceutically acceptable salt thereof

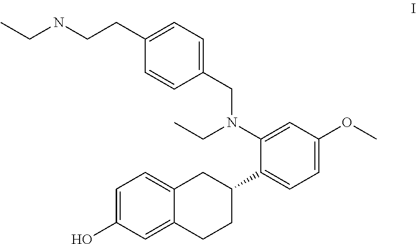

and an anti-estrogenic agent (e.g., an aromatase inhibitor, a SERM that is not the SERM of Formula I, a GNRH agonist, a GNRH antagonist or an estrogen receptor downregulator). The composition can be a pharmaceutical composition further comprising a pharmaceutically acceptable excipient.

In one embodiment, the composition comprises a compound of formula I, or a pharmaceutically acceptable salt thereof and an aromatase inhibitor. In a more particular embodiment, the composition is a pharmaceutical composition further comprising a pharmaceutically acceptable excipient.

In a particular embodiment of this invention, the composition comprises a compound of formula I, or a pharmaceutically acceptable salt thereof, and an aromatase inhibitor selected from the group consisting of: anastrozole, letrozole and exemestane.

In another particular embodiment of this invention, the composition comprises a compound of formula I, or a pharmaceutically acceptable salt thereof, and the aromatase inhibitor, anastrozole. In a more particular embodiment, the composition is a pharmaceutical composition further comprising a pharmaceutically acceptable excipient.

In another particular embodiment of this invention, the composition comprises a compound of formula I, or a pharmaceutically acceptable salt thereof, and the aromatase inhibitor, exemestane. In a more particular embodiment, the composition is a pharmaceutical composition further comprising a pharmaceutically acceptable excipient.

In yet another particular embodiment of this invention, the composition comprises a compound of formula I, or a pharmaceutically acceptable salt thereof, and the aromatase inhibitor, letrozole. In a more particular embodiment, the composition is a pharmaceutical composition further comprising a pharmaceutically acceptable excipient.

In another embodiment, the composition comprises the compound of formula I, or a pharmaceutically acceptable salt thereof, and a second selective estrogen receptor modulator (SERM). It is understood that the second SERM is not the compound of formula I. In a more particular embodiment, the composition is a pharmaceutical composition further comprising a pharmaceutically acceptable excipient.

In a particular embodiment, the composition comprises a compound of formula I or a pharmaceutically acceptable salt thereof and a selective estrogen receptor modulator selected from the group consisting of: tamoxifen, acolbolifene, arzoxifene, raloxifene, lasofoxifene, toremifene, pipindoxifene, and bazedoxifene.

In another particular embodiment, the composition comprises a compound of formula I, or a pharmaceutically acceptable salt thereof, and the selective estrogen modulator, tamoxifen. In a more particular embodiment, the composition is a pharmaceutical composition further comprising a pharmaceutically acceptable excipient.

In another particular embodiment of this invention, the composition comprises a compound of formula I, or a pharmaceutically acceptable salt thereof, and the selective estrogen receptor modulator, toremifene. In a more particular embodiment, the composition is a pharmaceutical composition further comprising a pharmaceutically acceptable excipient.

In yet another embodiment, the composition comprises the compound of formula I, or a pharmaceutically acceptable salt thereof, and a GNRH agonist or GNRH antagonist. In a more particular embodiment, the composition is a pharmaceutical composition further comprising a pharmaceutically acceptable excipient.

In a particular embodiment, of this invention, the composition comprises a compound of formula I, or a pharmaceutically acceptable salt thereof, and a GNRH agonist or a GNRH antagonist seleceted from the group consisting of: buserelin, goserelin, histrelin, leuprorelin, nafarelin, triptorelin, abarelix, cetrorelix and ganirelix.

In one embodiment of this invention the composition comprises the compound of formula I, or a pharmaceutically acceptable salt thereof, and an estrogen receptor downregulator. In a more particular embodiment, the composition is a pharmaceutical composition further comprising a pharmaceutically acceptable excipient.

In a particular embodiment, of this invention, the composition comprises a compound of formula I, or a pharmaceutically acceptable salt thereof, and the estrogen receptor downregulator fulvestrant.

The invention described herein also relates to a method of treating breast cancer in a subject in need thereof, comprising the combination administration to the subject of a first amount of a compound of formula I or a pharmaceutically acceptable salt thereof, and a second amount an anti-estrogenic agent, wherein the first and second amounts together comprise an effective amount.

In one embodiment, the method of treating breast cancer in a subject in need thereof comprises the combination administration to the subject of a first amount of a compound of formula I or a pharmaceutically acceptable salt thereof and a second amount an aromatase inhibitor, wherein the first and second amounts together comprise an effective amount.

In a particular embodiment, the aromatase inhibitor used in the method of treating breast cancer is selected from the group consisting of: anastrozole, letrozole and exemestane.

In another embodiment, the method of treating breast cancer in a subject in need thereof comprises the combination administration to the subject of a first amount of a compound of formula I or a pharmaceutically acceptable salt thereof and a second amount of a second selective estrogen receptor modulator, wherein the first and second amounts together comprise an effective amount. It is understood that the second selective estrogen receptor modulator is not the compound of formula I.

In a particular embodiment, the selective estrogen receptor modulator used in the method of treating breast cancer is selected from the group consisting of: tamoxifen, acolbolifene, arzoxifene, raloxifene, lasofoxifene, toremifene, pipindoxifene, and bazedoxifene.

In another embodiment, the method of treating breast cancer in a subject in need thereof comprises the combination administration to the subject of a first amount of a compound of formula I or a pharmaceutically acceptable salt thereof and a second amount of a GNRH agonist or GNRH antagonist, wherein the first and second amounts together comprise an effective amount.

In a particular embodiment, the GNRH agonist or GNRH antagonist used in the method of treating breast cancer is selected from the group consisting of: buserelin, goserelin, histrelin, leuprorelin, nafarelin, triptorelin, abarelix, cetrorelix and ganirelix.

In another embodiment, the method of treating breast cancer in a subject in need thereof comprises the combination administration to the subject of a first amount of a compound of formula I or a pharmaceutically acceptable salt thereof and a second amount of an estrogen receptor downregulator, wherein the first and second amounts together comprise an effective amount.

In a particular embodiment, the estrogen receptor downregulator used in the method of treating breast cancer is fulvestrant.

The invention described herein also relates to a method of treating breast cancer in a subject in need thereof, comprising administering to the subject an effective amount of a composition comprising a compound of formula I and an anti-estrogenic agent (e.g., an aromatase inhibitor, a SERM, a GNRH agonist, GNRH antagonist or an estrogen receptor downregulator). In a particular embodiment, the composition is a pharmaceutical composition further comprising a pharmaceutically acceptable excipient.

It is understood that any of the compositions and pharmaceutical compositions described herein can be administered in the method of treating breast cancer.

In another embodiment, this invention provides a method of treating vasomotor disturbances associated with anti-estrogen therapy comprising the administration of an effective amount of a compound of formula I or a pharmaceutically acceptable salt thereof to a subject treated with one or more of an anti-estrogneic agent. In a particular embodiment, the anti-estrogenic agent is selected from the group consisting of an aromatase inhibitor, a selective estrogen receptor modulator that is not the compound of formula I, a GNRH agonist, GNRH antagonist and an estrogen receptor downregulator.

In a particular embodiment, the subject is treated with an aromatase inhibitor. In a more particular embodiment, the aromatase inhibitor is selected from the group consisting of: anastrozole, letrozole and exemestane. In aother more particular embodiment, the aromatase inhibitor is anastrozole. In yet another more particular embodiment, the aromatase inhibitor is letrozole. In yet another more particular embodiment, the aromatase inhibitor is exemestane.

In another particular embodiment, the subject is treated with a selective estrogen receptor modulator. In a more particular embodiment, the selective estrogen receptor modulator is selected from: tamoxifen, acolbolifene, arzoxifene, raloxifene, lasofoxifene, toremifene, pipindoxifene, and bazedoxifene. In an even more particular embodiment, the selective estrogen receptor modulator is tamoxifen or toremifene. It is understood that the SERM with which the subject is treated is not the compound of formula I.

In yet another particular embodiment, the subject is treated with a GNRH agonist or GNRH antagonist. In a more particular embodiment, the GNRH agonist or GNRH antagonist is selected from the group consisting of: buserelin, goserelin, histrelin, leuprorelin, nafarelin, triptorelin, abarelix, cetrorelix and ganirelix.

In another particular embodiment, the subject is treated with an estrogen receptor downregulator. In a more particular embodiment, the estrogen receptor downregulator is fulvestrant.

In another particular embodiment, the vasomotor disturbance to be treated is selected from the group consisting of: hot flashes, night sweats, sleeplessness, anxiety and combinations thereof.

In another embodiment, this invention provides a method of treating a disease or disorder resulting from the loss of bone mineral density associated with anti-estrogen therapy comprising the administration of an effective amount of a compound of formula I or a pharmaceutically acceptable salt thereof to a subject treated with one or more of an anti-estrogenic agent. In a particular embodiment, the anti-estrogenic agent is selected from the group consisting of an aromatase inhibitor, a selective estrogen receptor modulator that is not the compound of formula I, a GNRH agonist, GNRH antagonist and an estrogen receptor downregulator.

In a particular embodiment, the subject is treated with an aromatase inhibitor. In a more particular embodiment, the aromatase inhibitor is selected from the group consisting of: anastrozole, letrozole and exemestane. In another more particular embodiment, the aromatase inhibitor is anastrozole. In yet another more particular embodiment, the aromatase inhibitor is letrozole. In yet another more particular embodiment, the aromatase inhibitor is exemestane.

In another particular embodiment, the subject is treated with a selective estrogen receptor modulator. In a more particular embodiment, the selective estrogen receptor modulator is selected from: tamoxifen, acolbolifene, arzoxifene, raloxifene, lasofoxifene, toremifene, pipindoxifene, and bazedoxifene. In an even more particular embodiment, the selective estrogen receptor modulator is tamoxifen or toremifene. It is understood that the SERM with which the subject is treated is not the compound of formula I

In yet another particular embodiment, the subject is treated with a GNRH agonist or GNRH antagonist. In a more particular embodiment, the GNRH agonist or GNRH antagonist is selected from the group consisting of: buserelin, goserelin, histrelin, leuprorelin, nafarelin, triptorelin, abarelix, cetrorelix and ganirelix.

In yet another particular embodiment, the subject is treated with an estrogen receptor downregulator. In a more particular embodiment, the estrogen receptor downregulator is fulvestrant.

In a particular embodiment, the disease or disorder resulting from loss of bone mineral density is selected from the group consisting of: osteoporosis, osteopenia and a bone fracture. In a more particular embodiment, the disease or disorder resulting from loss of bone mineral density is osteoporosis.

In another embodiment, this invention provides a method of treating musculoskeletal complaints or disorders associated with anti-estrogen therapy comprising the administration of an effective amount of a compound of formula I or a pharmaceutically acceptable salt thereof to a subject treated with one or more of an anti-estrogenic agent. In a particular embodiment, the anti-estrogenic agent is selected from the group consisting of: an aromatase inhibitor, a selective estrogen receptor modulator, a GNRH agonist, a GNRH antagonist and an estrogen receptor downregulator.

In a particular embodiment, the subject is treated with an aromatase inhibitor. In a more particular embodiment, the aromatase inhibitor is selected from the group consisting of: anastrozole, letrozole and exemestane. In aother more particular embodiment, the aromatase inhibitor is anastrozole. In yet another more particular embodiment, the aromatase inhibitor is letrozole. In yet another more particular embodiment, the aromatase inhibitor is exemestane.

In another particular embodiment, the subject is treated with a selective estrogen receptor modulator. In a more particular embodiment, the selective estrogen receptor modulator is selected from: tamoxifen, acolbolifene, arzoxifene, raloxifene, lasofoxifene, toremifene, pipindoxifene, and bazedoxifene. In an even more particular embodiment, the selective estrogen receptor modulator is tamoxifen or toremifene. It is understood that the SERM with which the subject is treated is not a compound of formula I.

In yet another particular embodiment, the subject is treated with an estrogen receptor downregulator. In a more particular embodiment, the estrogen receptor downregulator is fulvestrant.

In yet another particular embodiment, the subject is treated with a GNRH agonist or GNRH antagonist. In a more particular embodiment, the GNRH agonist or GNRH antagonist is selected from the group consisting of: buserelin, goserelin, histrelin, leuprorelin, nafarelin, triptorelin, abarelix, cetrorelix and ganirelix.

In another embodiment, this invention provides a method of treating the uterine side effects associated with tamoxifen therapy, comprising the administration of an effective amount of compound of formula I to a subject treated with tamoxifen.

In another embodiment, this invention provides a method of treating the uterine side effects associated with toremifene therapy, comprising the administration of an effective amount of a compound of formula I to a subject treated with toremifene.

In another embodiment, this invention describes the use of a compound of formula I, or a pharmaceutically acceptable salt thereof with at least one anti-estrogenic agent for the manufacture of a medicament for treating breast cancer. The anti-estrogenic agent can be selected from the group consisting of: an aromatase inhibitor, a GNRH agonist, a GNRH antagonist, a SERM and an estrogen receptor downregulator.

In another embodiment, this invention describes the use of a compound of formula I, or a pharmaceutically acceptable salt thereof with at least one anti-estrogenic agent selected from the group consisting of: tamoxifen, raloxifene, bazedoxifene, pipindoxifene, fulvestrant, acolbolifene, toremifene, exemestane, arimidex and letrozole for the manufacture of a medicament for treating breast cancer.

In yet another embodiment, this invention describes the use of a compound of formula I, or a pharmaceutically acceptable salt thereof in the manufacture of a medicament for treating vasomotor disturbances associated with the administration of an anti-estrogenic agent.

In another embodiment, this invention describes the use of a compound of formula I, or a pharmaceutically acceptable salt thereof for the manufacture of a medicament for treating vasomotor disturbances associated with the administration of at least one of tamoxifen, raloxifene, bazedoxifene, pipindoxifene, fulvestrant, acolbolifene, toremifene, exemestane, arimidex and letrozole.

### DETAILED DESCRIPTION OF THE INVENTION

The use of certain anti-estrogenic agents for the treatment of estrogen-dependent breast cancer is well-established. In general, the agent classes that have been found useful for treating these diseases are aromatase inhibitors (e.g. letrozole, anastrozole, aromasin), SERMs (e.g. tamoxifen, raloxifene, toremifene), estrogen receptor down-regulators (e.g.fulvestrant), GNRH agonists (buserelin, goserelin, histrelin, leuprorelin, nafarelin, triptorelin) and GNRH antagonists (abarelix, cetrorelix and ganirelix). While each of these classes of anti-estrogenic agents has utility in the treatment of estrogen-dependent breast cancer, they all have side effects associated with their effect at the estrogen receptor. For example, the use of aromatase inhibitors (agent that inhibits the conversion of androstenedione to estrone or testosterone to estradiol) is associated with joint pain, bone pain and/or muscle pain (musculoskeletal complaints), bone loss (e.g. osteoporosis) and vasomotor symptoms (e.g. hot flashes). SERMs clinically used to treat breast cancer are associated with increased vasomotor symptoms as well. The SERMs tamoxifen and toremifene are additionally associated with uterine stimulation. Clinically, these can be difficult to treat side effects, because the source of the side effect is the alteration in estrogen function necessary for treatment.

For example, hot flashes are a common effect of women undergoing estrogen withdrawal whether through a natural experience of menopause or inhibition of estrogen formation as occurs with treatment using an aromatase inhibitor. However, whereas in the case of menopause, a woman may be treated with an estrogen containing medicine such as occurs with hormone replacement therapy, such a treatment is contraindicated in women receiving aromatase inhibitors, SERMs, GNRH agonists, GNRH antagonists or estrogen receptor down-regulators since treatment is focused on inhibiting agonist signaling thru the estrogen pathway as it pertains to the estrogen-dependent breast cancer being treated. As a result, the side effects of the treatment are difficult to treat since many estrogen withdrawal side effects, such as hot flash, cannot be adequately treated without resorting to contraindicated hormonal methods. As a result, current treatments include ignoring the side effect or resorting to less effective treatment modalities. For example, while it is well-known that certain SSRI's (e.g. paroxetine) or SNRI's (e.g. venlafaxine) are capable of alleviating hot flashes, it is generally accepted that they do not have the efficacy of estrogen-containing treatments and may have side effects as well (e.g. sexual dysfunction, dry mouth, dizziness).

In the published patent application, US2006/011634, the compound of formula I was disclosed as example 736 and more recently the compound of formula I was also disclosed in WO2008/002490, both of which are herein incorporated by reference in their entirety. Of particular interest, it was reported that the compound of formula I could be used to treat vasomotor symptoms despite the fact that it demonstrated no estrogenic stimulation on the rat uterus (unlike tamoxifen and raloxifene both of which did stimulate the uterus in the reported results), and even could antagonize the estrogenic effect of estradiol on the uterus and bring it back to control levels.

The present invention makes further use of the compound of formula I in combination therapies for the treatment of breast cancer comprising administration of a compound of formula I and an anti-estrogenic agent (e.g., an aromatase inhibitor, a SERM that is not the SERM of Formula I, a GNRH agonist, a GNRH antagonist, or an estrogen receptor downregulator) and in compositions (e.g., pharmaceutical compositions) comprising a compound of Formula I and an anti-estrogenic agent. This invention also makes use of the compound of formula I in a method of treating the side effects (e.g., vasomotor disturbances, osteoporosis and musculoskeletal complaints) associated with anti-estrogen therapy in a subject treated with one or more anti-estrogenicic agents (e.g., an aromatase inhibitor, a SERM that is not the SERM of Formula I, a GNRH agonist, a GNRH antagonist, or an estrogen receptor downregulator).

For the purposes of this disclosure, the therapeutic utility of these compounds includes “treating” a human, where treating is understood to include treating, preventing, or ameliorating the symptoms associated with, or reducing the incidence of, reducing the pathogenesis of, facilitating the recovery from or delaying the onset of the syndrome, illness, malady or condition being considered.

As used herein, an effective amount refers to an amount sufficient to elicit the desired biological response. In the present invention, the desired biological response is treating, preventing, or ameliorating the symptoms associated with, or reducing the incidence of, reducing the pathogenesis of, facilitating the recovery from or delaying the onset of the syndrome, illness, malady or condition being considered. For example, in the combination therapies (administration of a first amount of a compound of formula I and a second amount of an anti-estrogenic agent) the desired biological response is treating breast cancer. In the monotherapies, using a compound of formula I, the desired biological response is a reduction in at least one side effect associated with the administration of an anti-estrogenic agent in a subject treated with the anti-estrogenic agent.

An effective amount can be achieved in the combination therapies of the invention by coadministering a first amount of a compound of formula I and a second amount of an anti-estrongenic agent (e.g., an aromatase inhibitor, a SERM or an estrogen receptor downregulator) wherein the first and second amounts together comprise an effective amount. For example, in one embodiment, the compound of formula I and the anti-estrogenic agent can each be administered in an effective amount (i.e., an amount which would be effective if administered alone). In another embodiment, the compound of formula I and the anti-estrogenic agent can be administered in an amount for which each alone does not provide a therapeutic effect (a sub-therapeutic dose). In yet another embodiment, the compound of formula I can be administered in an effective amount, while the anti-estrogenic agent can be administed in a sub-therapeutic dose. In still another embodiment, the compound of formula I can be administed in a sub-therapeutic dose, while the anti-estrogneic agent can be administed in an effective amount.

As used herein, an anti-estrogenic agent is any agent which decreases the amount of estrogen present in the subject being treated. Suitable examples of anti-estrogenic agents include aromatase inhibitors, GNRH agonists, GNRH antagonists, SERMs and estrogen receptor downregulators. As used herein, anti-estrogen therapy is therapy which includes the administration of an anti-estrogenic agent.

Aromatase inhibitors are agents that inhibits the conversion of androstenedione to estrone or testosterone to estradiol. Examples of aromatase inhibitors include, but are not limited to, anastrozole, exemestane and letrozole.

SERMs are agents that compete with endogenous estrogens (e.g., 17β-estradiol), thereby blocking the proliferative effects of these endogenous estrogens. Examples of SERMs include, but are not limited to, tamoxifen, acolbolifene, arzoxifene, raloxifene, lasofoxifene, toremifene, pipindoxifene, and bazedoxifene. It is understood that the compound of formula I although a selective estrogen receptor modulator, is not included within the definition of SERM as used herein.

Estrogen receptor downregulators are compounds that facilitate the degradation of the estrogen receptor. An example of an estrogen receptor downregulator is fulvestrant.

GNRH agonists and GNRH antagonists are agents that shut down the endogenous production of sex hormones (e.g. estradiol and testosterone). Examples of GNRH agonists and GNRH antagonists include, but are not limited to, buserelin, goserelin, histrelin, leuprorelin, nafarelin, triptorelin, abarelix, cetrorelix and ganirelix.

As used herein, side effects associated with the administration of an anti-estrogenic agent or anti-estrogen therapy means side effects which can result from treatment with an anti-estrogenic agent (e.g., an aromatase inhibitor, a SERM, a GNRH agonist, a GNRH antagonist, or an estrogen receptor downregulator). The side effects can be experienced while the subject is being treated with the anti-estrogenic agent, following treatment or both during and after treatment. As such, a subject treated with an anti-estrogenic agent includes subjects undergoing treatment and subjects who have finished treatment, but still experience the side effects of the treatment.

Musculoskeletal complaints or disorders are a common side effect of anti-estrogenic agents, for example, aromatase inhibitors. The compound of formula I can be particularly useful for treating said musculoskeletal complaints or disorders associated with the administration of anti-estrogenic agents (e.g., aromatase inhibitors, a GNRH agonist, a GNRH antagonist, SERMs and estrogen receptor downregulators). Without wishing to be strictly bound by these definitions—musculoskeletal complaints or disorders are of a complex etiology, but often present the subject with muscle, bone or joint pain, stiffness or swelling. These are the general symptoms, but the cause (and thus the primary malady being treated) includes osteoarthritis, rheumatoid arthritis, tendonitis, bursitis, fibromyalgia, adhesive capsulitis, carpal tunnel syndrome, gout, chondrocalcinosis, giant cell arteritis, polymyalgia rheumatica, lupus, Sjorgen's syndrome, polymyositis and dermatomyositis.

The combination methods and compositions (e.g., pharmaceutical compositions) of this invention include in their utility the treatment of breast cancer. Unlike tamoxifen, which appears to not improve (and possibly decreases) the efficacy of the aromatase inhibitor anastrozole in the adjuvant setting (measuring breast cancer recurrence rates) in the widely published ATAC trial, it is believed that the compound of formula I of this invention will improve upon the efficacy of the primary treatment outcomes (reduction in breast cancer incidences and severity whether in primary treatment or adjuvant setting). The improved efficacy of the compound of formula I is believed to be possible through its very low proliferative signaling on the estrogen receptor as determined by reduced stimulation on other estrogen responsive tissues such as the uterus. Tamoxifen's basal signaling thru the estrogen receptor alpha is a possible reason for the compound's failure to improve upon the efficacy of anastrozole and possibly even makes it less efficacious. Lowering that signaling with a more complete antagonist could improve the combination efficacy at the same time as reducing the many side effects of the aromatase inhibitor thru the estrogen-agonist like effect in certain other tissues (bone, CNS, musculoskeletal).

Vasomotor disturbances are common side effects when undergoing estrogen ablation therapy whether through use of an aromatase inhibitor, estrogen receptor down-regulator, a GNRH agonist, a GNRH antagonist, or a SERM. The compound of formula I can be particularly useful for treating vasomotor disturbances associated with administration of an anti-estrogenic agent (e.g., an aromatase inhibitor, a GNRH agonist, a GNRH antagonist, a SERM or an estrogen receptor downregulator) without compromising and possibly even increasing the efficacy of the treatment with the anti-estrogenic agent in some cases. Unlike estrogens which are contraindicated with estrogen ablation therapy using an aromatase inhibitor, SERM, a GNRH agonist, a GNRH antagonist, or estrogen receptor down-regulator is being used, the estrogen receptor modulator of formula I is believed to be useful in combination without negatively impacting the therapy. The vasomotor symptoms that are treatable are the same type and nature as those which can be treated with estrogen. For example, hot flashes, sweats, night sweats and mood disturbances associated with any or all of the thermoregulatory disorders are vasomotor disturbances which are treatment objects of this invention.

The compouond of formula I is also useful for the treatment of diseases and disorders resulting from bone density loss (e.g, osteoporosis, osteopenia and bone fractures) associated with the administration of an anti-estrogenic agent. Of particular interest and utility is the use of a compound of formula I in a subject being treated with an aromatase inhibitor. The advantage of treating the bone density loss associated with the administration of the anti-estrogenic agent (e.g., an aromatase inhibitor) relates to the fact that aromatase inhibitors cause bone loss through estrogen deprivation, and that replacing the lost estrogen with a typical estrogen agonist (e.g. estradiol) would defeat the purpose of the therapy under normal circumstances. However, the compound of formula I is a very unique estrogen receptor modulator due to its ability to bind the estrogen receptor and protect from bone loss and hot flash while at the same time, not allowing orgreatly reducing the proliferative signaling normally enhanced when using a typical estrogen agonist (e.g. estradiol). As a result, the compound of formula I is uniquely positioned for the treatment of side effects associated with the administration of anti-estrogenic agents, in particular aromatase inhibitors. That is, the invention relates to a method of treating diseases and disorders resulting from loss of bone mineral density (e.g., osteoporsosis, osteopenia and bone fractures) in a subject being treated with an anti-estrogenic agent.

When the compound of formula I is referred to, it should be appreciated that the compound may also be used as a salt. Some representative (non-limiting) acid addition salts include hydrochloride, hydrobromide, hydroiodide, acetate, benzenesulfonate, mesylate, besylate, benzoate, tosylate, citrate, tartrate, sulfate, bisulfate, lactate, maleate, mandelate, valerate, laurate, caprylate, propionate, succinate, phosphate, salicylate, napsylate, nitrate, tannate, resorcinate and the like, including multiprotic salts as well as mixtures of the acid addition salts.

The compound formula I, when used in the combination methods of this invention may be co-formulated or co-administered wherein said co-administration does not require dosing at exactly the same time but rather indicates that the patient is undergoing treatment with one or more of the additional agents during the timeframe of treatment with the compound of this invention. Thus, the compound of formula I can be administered concomitantly, sequentially or separately from aromatase inhibitors, SERMs, GNRH agonists, GNRH antagonists or estrogen receptor down regulators of the combination therapy. By way of non-limiting example, the compound of this invention may be combined into a pill, capsule or a tablet containing a SERM, an aromatase inhibitor, a GNRH agonists, a GNRH antagonists or an estrogen receptor downregulator for use in once daily or multiple daily oral administrations. Alternatively, the compound of this invention maybe useful for the methods of this invention wherein the combination regimen consists of different dosing schedule for each of the drugs in the combination. For example, the estrogen receptor downregulator fulvestrant is typically dosed by once-monthly intramuscular injection (either one injection or two). A combination dosing regimen with fulvestrant and the compound of formula I might consist of the once monthly dosing of fulvestrant and the daily oral dosing of the compound of formula I.

The dosages of the combination components of the combination therapy and of the compound of formula I in the monotherapy treatment of the side effects associated with the administration of anti-estrogenic agents can be adjusted as deemed necessary by the subject or preferably by the subject in consultation with a qualified practitioner of medicine. Dosing of the combinations of the methods of this invention can take place by multiple routes. The dosing schedule and amounts of the combination components and of the compound of formula I in the monotherapy treatments of side effects are dependent not only on the particular subject's weight, sex, age, therapy contemplated, etc., but also by the route of administration. By way of non-limiting example, the combination therapies of this invention may be dosed by the oral route in a once daily, twice daily, three times daily or more than three times per day depending on the particular needs of that subject, the formulation of the drug, etc. The dosage of the compound of formula I will typically be from about 0.01 mg to about 500 mg of drug per daily dosage, such as from about 0.1 mg to about 250 mg, for example, from about 1 mg to about 150 mg, or from about 5 mg to about 100 mg.

The combination components or the compound of formula I of the monotherapy treatment of the side effects associated with the administration of an anti-estrogenic agent can be administered by any method known to one of skill in the art such as orally, bucally, intravenously, subcutaneously, intramuscularly, transdermally, intradermally, intravascularly, intranasally, sublingually, intracranially, rectally, intratumorally, intravaginally, intraperitonealy, pulmonary, ocularly and intratumorally, or when applicable a combination of more than one of the modes of administration (e.g. fulvestrant by once-monthly injection together with daily oral administration of the compound of formula I).

When administered, the combinations of this invention or the compound of formula I alone in the monotherapy treatment of side effects associated with the aministration of anti-estrogenic agents can be given once daily or with multiple daily doses such as twice per day, three times per day and four times per day, or in the case of combination therapy one of the components maybe given according to one schedule and the other component to a different schedule. The most common scheduled use of the combinations of this invention will use the aromatase inhibitor, a GNRH agonist, a GNRH antagonist, SERM or estrogen receptor down regulator according to its normal dosing schedule and dose the compound of formula I once per day, most typically by the oral route. Depending on the needs of the patient, the compound of formula I can be used in both combination therapy and in monotherapy for the treatment of side effects at a certain dose for a certain length of time however necessary to fulfill that patient's needs. Thus, by way of non-limiting example, the compound of formula I might be used once a day at a dose of 20 mg for the alleviation of hot flashes being experienced by a woman taking an aromatase inhibitor for the treatment of breast cancer. That patient may decide to undergo a fixed schedule with the compound of formula I or alternatively, may take the compound of formula I on an on-again, off-again basis depending on the perceived severity of her symptoms. In contrast, another patient is undergoing combination therapy taking the compound of formula I in a 50 mg dose together with an aromatase inhibitor for the primary purpose of increasing the effectiveness of the cancer treatment. In such a situation, that patient may be instructed to take the compound of formula I concomitantly with the aromatase inhibitor, on a daily basis, for as long as the aromatase inhibitor is being given. Neither of these examples are meant to limit the invention but rather to illustrate that the dosages and regimens of the compound of formula I are to be tailored according to the specific needs of the patients—examples will be provided in the examples section of this invention description.

In one embodiment of this invention, the compound of formula I alone or in combination with an anti-estrogenic agent can be administered orally where it can be formulated for solid dosage administration or liquid dosage administration. Solid dosage administration can be in the form of a tablet, granule, capsule, pill, pellet, powder and the like. Liquid dosage formulations include syrups, solutions, gels, suspensions, elixirs, emulsions, colloids, oils, and the like.

As mentioned previously, the compound of formula I alone or in combination with an anti-estrongenic agent can be solids and when present as solids, can be of defined particle size—it is sometimes preferable to administer the compound with a certain particle size—a particle size with a preferred range where the average mean particle size diameter is under 100 microns, or 75 microns, or 50 microns, or 35 microns, or 10 microns or 5 microns.

Solid dosage formulations will comprise at least a compound of formula I of this invention together with one or more pharmaceutical excipients. In the case of combination therapy, the solid dosage formulation can comprise a compound of formula I, an anti-estrogenic agent and a pharmaceutically acceptable excipient. Those excipients are known to one of skill in the art and include, by way of non-limiting example diluents (monosaccharides, disaccharides and polyhydric alcohols including starch, mannitol, dextrose, sucrose, microcrystalline cellulose, maltodextrin, sorbitol, xylitol, fructose and the like), binders (starch, gelatin, natural sugars, gums, waxes and the like), disintegrants (alginic acid, carboxymethylcellulose (calcium or sodium), cellulose, crocarmellose, crospovidone, microcrystalline cellulose, sodium starch glycolate, agar and the like), acidic or basic buffering agents (citrates, phoshphates, gluconates, acetates, carbonates, bicarbonates and the like), chelating agents (edetic acid, edetate calcium, edetate disodium and the like), preservatives (benzoic acid, chlorhexidine gluconate, potassium benzoate, potassium sorbate, sorbic acid, sodium benzoate and the like), glidants and lubricants (calcium stearate, oils, magnesium stearate, magnesium trisilicate, sodium fumarate, colloidal silica, zinc stearate, sodium oleate, stearic acid, and the like), antioxidants and/or preservatives (tocopherols, ascorabtes, phenols, and the like) and acidifying agents (citric acid, fumaric acid, malic acid, tartaric acid and the like) as well as coloring agents, coating agents, flavoring agents, suspending agents, desiccants, humectants and other excipients known to those of skill in the art.

The solid dosage formulations of this invention can be prepared in different forms including most commonly, tablets and capsules. The tablets can be formulated by a wide variety of methods known to one of skill in the art including, for example, preparing a dry powder mixture of the drug substance in combination with one or more of the excipients granulating the mixture and pressing to together into a tablet and optionally coating the tablet with an enteric or non-enteric coating. The final coat typically includes a light protective pigment such as titanium oxide and a shellac or wax to keep the tablet dry and stable. While not intending to be limited by theory or example, in some instances it might be preferred to prepare the tablets by wet granulating the drug with one or more of the excipients and then extruding the granulated material.

The solid dosage forms of this invention also include capsules wherein the drug is enclosed inside the capsule either as a powder together with optional excipients or as granules containing one or more excipients together with the drug and wherein the granule in turn can be optionally coated, for example, enterically or non-enterically.

In certain embodiments of this invention, the solid dosage formulations are formulated in a sustained release formulation. Such formulations are known to those of skill in the art and generally rely on the co-formulation of the drug/active with one or more matrix forming substances that slow the release of the incorporated active (e.g., a compound of formula I alone or in combination with an anti-estrogenic agent) thus extending the lifetime of the formulation in the digestive track and thereby extending the compounds half-life. Some non-limiting matrix forming substances include hydroxypropyl methylcellulose, carbopol, sodium carboxymethylcellulose and the like.

In some embodiments of this invention, the compounds are formulated for delivery other than via a solid oral dosage form. For example, in certain instances it might be preferable to deliver via a pulmonary route. A pulmonary route of administration typically means that the components of the combination therapy or a compouond of formula I is inhaled into the lung where it is absorbed into the circulation. Such a route of administration has the advantage of avoiding a first pass liver effect. Suitable formulation for pulmonary delivery can be accomplished by micronizing the actives to a very fine size particle, typically with a mean average diameter of less than 20 microns, or less than 10 microns or between 2 and 5 microns. The powder may then be inhaled by itself or more likely mixed with one or more excipients such as lactose or maltose. The powder can then be inhaled in a dry powder inhaling device either once or multiple times per day depending on the particular compound and the patient's need. Other types of pulmonary dosage forms are also embraced by this invention. In an alternative to the dry powder delivery, the active may be suspended in an aerosolizing medium and inhaled as a suspension through a meter dosed inhaler or a nebulizer.

The components of the combination therapy of the compound of formula I can be formulated for transdermal delivery. A wide variety of transdermal options can be used. For example, the components of the combination therapy or the compound of formula I can be formulated for passive diffusion patches preferably embedded in a matrix that allows for slow diffusion of the actives into the treated subject's circulation. For this purpose, the active is preferably dissolved or suspended in solvents including by way of non-limiting examples one or more of ethanol, water, propylene glycol, and Klucel HF. In some instances, a polymer matrix (e.g. acrylate adhesive) will comprise the bulk of the transdermal formulation. In some instances, the transdermal formulations maybe designed to be compatible with alternate transdermal delivery technologies. For example, some transdermal technologies achieve greater and/or more consistent delivery by creating micropores in the skin using radio frequency, heat, ultrasound or electricity. In some cases, the compounds of this invention can be used with microneedle technology wherein the compound is loaded into very small needles which do not need to penetrate the dermis to be effective.

### EXAMPLES OF THE INVENTION

### 1)—Compound Formula I Plus Aromatase Inhibitor

The methods and compositions of this invention provide specifically for a compound of formula I for use together with an aromatase inhibitor. In particular, the methods are combined treatment of breast cancer and of at least one symptom associated with the use of an aromatase inhibitor selected from the group consisting of: vasomotor symptoms; osteoporosis; and musculoskeletal complaints. Some prophetic examples of combinations of the compound of formula I with different aromatase inhibitors or tamoxifen are provided below:

### Example 1

Compound formula 1 50 mg
Letrozole 2.5 mg
Microcrystalline Cellulose 38.5 mg
Starch 35 mg
SiO₂ 3 mg
Mannitol 23 mg

The ingredients in Example 1 are mixed together and sieved to homogeneity. The resulting powder is compressed with a punch and the resulting tablets spray coated with Opadry® II blue with an increase of tablet weight of 4% yielding a total tablet weight of approximately 158 mg.

### Example 2

Compound formula 1 50 mg
Anastrozole 1 mg
Microcrystalline Cellulose 27.5 mg
Starch 25 mg
SiO₂ 1 mg
Mannitol 31 mg

The ingredients in Example 2 are mixed together and sieved to homogeneity. The resulting powder is compressed with a punch and the resulting tablets spray coated with Opadry® II red with an increase of tablet weight of 4% yielding a total tablet weight of approximately 141 mg.

### Example 3

Compound formula 1 50 mg
Exemestane 25 mg
Microcrystalline Cellulose 37.5 mg
Starch 30 mg
SiO₂ 2.5 mg
Mannitol 26 mg

The ingredients in Example 3 are mixed together and sieved to homogeneity. The resulting powder is compressed with a punch and the resulting tablets spray coated with Opadry® II green with an increase of tablet weight of 4% yielding a total tablet weight of approximately 178 mg.

### 2)—Compound Formula I Plus SERM

The methods and compositions of this invention provide specifically for a compound of formula I for use together with a SERM that is different from the compound of formula I. In particular, the methods are a combined treatment of breast cancer and of at least one symptom associated with the use of a SERM other than the compound of formula I selected from the group consisting of: vasomotor symptoms and uterine effects. Some examples of combinations of the compound of formula I with different SERMs are provided below:

### Example 4

Compound formula 1 50 mg
Tamoxifen citrate 30.4 mg
lactose, NF 200 mg
microcrystalline cellulose 100 mg
starch 25 mg
sodium lauryl sulfate 10 mg
ascorbic acid 5 mg
SiO₂ 1 mg
magnesium stearate 3 mg

The ingredients in Example 4 are mixed together and sieved. The resulting powder is compressed with a punch and the resulting tablets spray coated with Opadry® I white with an increase of tablet weight of 4% yielding a total tablet weight of approximately 441 mg.

### Example 5

Compound formula 1 50 mg
toremifene citrate 88.5 mg
lactose, NF 200 mg
microcrystalline cellulose 100 mg
starch 25 mg
sodium lauryl sulfate 10 mg
ascorbic acid 5 mg
SiO₂ 1 mg
magnesium stearate 3 mg

The ingredients in Example 5 are mixed together and sieved. The resulting powder is compressed with a punch and the resulting tablets spray coated with Opadry® yellow with an increase of tablet weight of 4% yielding a total tablet weight of approximately 502 mg.

### Example 6

A postmenopausal female patient is being treated in the adjuvant setting for breast cancer with the SERM tamoxifen citrate (dosage of 20 mg tamoxifen free base). She is experiencing hot flashes as a result of her treatment. In accordance with the methods of this invention, she is dosed once daily with a 20 mg formulation of the compound of formula I. She reports a partial alleviation of her symptoms so the dosage is adjusted to a 50 mg formulation of the compound of formula I where she reports an almost complete alleviation of her hot flushes.

While this invention has been particularly shown and described with references to example embodiments thereof, it will be understood by those skilled in the art that various changes in form and details may be made therein without departing from the scope of the invention encompassed by the appended claims.

1. A composition comprising a compound of formula I 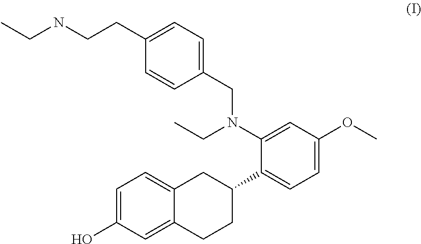
or pharmaceutically acceptable salt thereof and an anti-estrogenic agent selected from the group consisting of: an aromatase inhibitor, a selective estrogen receptor modulator, a GNRH agonist, a GNRH antagonist, and an estrogen receptor downregulator.

2. The composition of claim 1, wherein the anti-estrogenic agent is an aromatase inhibitor.

3. The composition of claim 2, wherein the aromatase inhibitor is selected from the group consisting of: anastrozole, letrozole and exemestane.

4. The composition of claim 3, wherein the the aromatase inhibitor is anastrozole.

5. The composition of claim 3, wherein the aromatase inhibitor is exemestane.

6. The composition of claim 3, wherein the aromatase inhibitor is letrozole.

7. The composition of claim 1, further comprising a pharmaceutically acceptable excipient.

8. The composition of claim 1, wherein the anti-estrogenic agent is a selective estrogen receptor modulator.

9. The composition of claim 8, wherein the selective estrogen receptor modulator is selected from the group consisting of: tamoxifen, acolbolifene, arzoxifene, raloxifene, lasofoxifene, toremifene, pipindoxifene, and bazedoxifene.

10. The composition of claim 9, wherein the selective estrogen modulator is tamoxifen.

11. The composition of claim 9, wherein the selective estrogen receptor modulator is toremifene.

12. The composition of claim 9, wherein the selective estrogen receptor modulator is lasofoxifene.

13. The composition of claim 8, further comprising a pharmaceutically acceptable excipient.

14. The composition of claim 1, wherein the anti-estrogenic agent is and an estrogen receptor downregulator.

15. The composition of claim 14, wherein the estrogen receptor down regulator is fulvestrant.

16. The composition of claim 14 further comprising a pharmaceutically acceptable excipient.

17. The composition of claim 1, wherein the anti-estrogenic agent is a GNRH agonist or GNRH antagonist.

18. The composition of claim 17 wherein the GNRH agonist or GNRH antagonist is selected from the group consisting of: buserelin, goserelin, histrelin, leuprorelin, nafarelin, triptorelin, abarelix, cetrorelix and ganirelix.

19. The composition of claim 17, further comprising a pharmaceutically acceptable excipient.

20-30. (canceled)

31. A method of treating vasomotor disturbances associated with anti-estrogen therapy comprising administering an effective amount of a compound of formula I 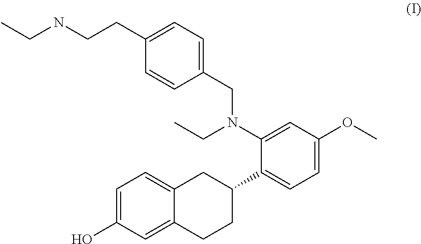
or a pharmaceutically acceptable salt thereof to a subject treated with one or more of an anti-estrogenic agent.

32. The method of claim 31, wherein the anti-estrogenic agent is selected from the group consisting of an aromatase inhibitor, a selective estrogen receptor modulator, a GNRH agonist, a GNRH antagonist and an estrogen receptor downregulator.

33. The method of claim 32, wherein the subject is treated with an aromatase inhibitor.

34. The method of claim 33, wherein the aromatase inhibitor is selected from the group consisting of: anastrozole, letrozole and exemestane.

35. The method of claim 34, wherein the aromatase inhibitor is anastrozole.

36. The method of claim 34, wherein the aromatase inhibitor is letrozole.

37. The method of claim 34, wherein the aromatase inhibitor is exemestane.

38. The method of claim 32, wherein the subject is treated with a selective estrogen receptor modulator.

39. The method of claim 38, wherein the selective estrogen receptor modulator is selected from the group consisting of: tamoxifen, acolbolifene, arzoxifene, raloxifene, lasofoxifene, toremifene, pipindoxifene, and bazedoxifene.

40. The method of claim 39, wherein the selective estrogen receptor modulator is tamoxifen or toremifene.

41. The method of claim 32, wherein the subject is treated with an estrogen receptor downregulator.

42. The method of claim 41 where the estrogen receptor downregulator is fulvestrant.

43. The method of claim 32, wherein the GNRH agonist or GNRH antagonist is selected from the group consisting of: buserelin, goserelin, histrelin, leuprorelin, nafarelin, triptorelin, abarelix, cetrorelix and ganirelix.

44. The method of claim 31, wherein the vasomotor disturbance is selected from the group consisting of: hot flashes, night sweats, sleeplessness, anxiety and combinations thereof.

45-73. (canceled)
